(19) United States
(12) Patent Application Publication
(10) Pub. No.: US 2025/0000823 A1
(43) Pub. Date: Jan. 2, 2025

(54) METHODS AND COMPOSITIONS FOR TREATING CONDITIONS ASSOCIATED WITH CENTRAL HYPOVENTILATION

(71) Applicant: APNIMED, INC. (DELAWARE), Cambridge, MA (US)

(72) Inventors: Luigi TARANTO-MONTEMURRO, Brookline, MA (US); David P. WHITE, Denver, CO (US); Ronald FARKAS, Clarksville, MD (US); Lawrence G. MILLER, Cambridge, MA (US)

(21) Appl. No.: 18/709,055

(22) PCT Filed: Nov. 10, 2022
(86) PCT No.: PCT/US2022/049483

§ 371 Date: May 10, 2024

Related U.S. Application Data
(60) Provisional application No. 63/278,324, filed on Nov. 11, 2021.
(60) Provisional application No. 63/305,305, filed on Feb. 1, 2022.

(51) Int. Cl. A61K 31/138; A61K 31/433; A61P 11/00

(52) U.S. Cl. CPC A61K 31/138; A61K 31/433; A61P 11/00

(57) ABSTRACT

Pharmaceutical compositions comprising (i) a norepinephrine reuptake inhibitor (NRI) and (ii) a carbonic anhydrase inhibitor (CAI) and methods of treating conditions associated with central hypoventilation are described herein.

### CROSS-REFERENCE TO RELATED APPLICATIONS

The present application claims priority to and benefit of U.S. provisional application nos. 63/278,324, filed Nov. 11, 2021, and 63/305,305, filed Feb. 1, 2022, the entire contents of each of which are incorporated herein by reference.

### TECHNICAL FIELD

The present invention provides pharmaceutical compositions comprising (i) a norepinephrine reuptake inhibitor (NRI) and (ii) a carbonic anhydrase inhibitor, and methods of treating conditions associated with central hypoventilation.

### BACKGROUND

Obesity hypoventilation syndrome (OHS) is a condition associated with obesity in which patients fail to breathe rapidly or deep enough, resulting in low oxygen levels and high blood CO₂ levels. Untreated OHS is associated with significant morbidity.

### SUMMARY

One aspect of the present invention provides a method of treating a subject having a condition associated with central hypoventilation, the method comprising administering to a subject in need thereof an effective amount of (i) a norepinephrine reuptake inhibitor (NRI) and (ii) a carbonic anhydrase inhibitor (CAI).

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is reboxetine or a pharmaceutically acceptable salt thereof. In some embodiments, the NRI is atomoxetine or a pharmaceutically acceptable salt thereof. In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. In some embodiments, the NRI, such as atomoxetine or a pharmaceutically acceptable salt thereof, is administered at a dose of from about 20 to about 200 mg. In some embodiments, the NRI, such as atomoxetine or a pharmaceutically acceptable salt thereof, is administered at a dose of from about 25 to about 100 mg. In some embodiments, the CAI, such as acetazolamide, is administered at a dosage of from about 150 mg to about 750 mg. In some embodiments, the carbonic anhydrase inhibitor, such as acetazolamide, is administered at a dosage of about 500 mg. In some embodiments, the carbonic anhydrase inhibitor, such as acetazolamide, is administered at a dosage of about 250 mg. In some embodiments, the dose is a daily dose, i.e., administered once per day. In some embodiments, the dose is a twice-daily dose, i.e., administered in two administrations per day (e.g., once in the morning and once before bedtime). In some embodiments, the NRI and CAI are administered as separate compositions. In some embodiments, the NRI and CAI are administered in a single composition. In some embodiments, the separate compositions or single composition are an oral administration form. In some embodiments, the oral administration form is a syrup, pill, tablet, troche, capsule, or patch. In some embodiments, the condition associated with central hypoventilation is obesity hypoventilation syndrome (OHS) or obesity-related sleep hypoventilation (ORSH). In some embodiments, the condition associated with central hypoventilation is obesity hypoventilation syndrome (OHS). In some embodiments, the condition associated with central hypoventilation is obesity-related sleep hypoventilation (ORSH).

Another aspect of the present invention provides a pharmaceutical composition comprising (i) a norepinephrine reuptake inhibitor (NRI) and (ii) a carbonic anhydrase inhibitor (CAI), and (iii) a pharmaceutically acceptable carrier.

Embodiments of this aspect of the invention may include one or more of the following optional features. In some embodiments, the NRI is a norepinephrine selective reuptake inhibitor (NSRI). In some embodiments, the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof. In some embodiments, the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof. In some embodiments, the NRI is reboxetine or a pharmaceutically acceptable salt thereof. In some embodiments, the NRI is atomoxetine or a pharmaceutically acceptable salt thereof. In some embodiments, the CAI is selected from the group consisting of acetazolamide, dichlorophenamide, dorzolamide, brinzolamide, methazolamide, zonisamide, ethoxzolamide, topiramate, sultiame, and any combination thereof, including pharmaceutically acceptable salts thereof. In some embodiments, the CAI is acetazolamide or a pharmaceutically acceptable salt thereof. In some embodiments, the NRI, such as atomoxetine or a pharmaceutically acceptable salt thereof, is present in an amount of from about 20 to about 200 mg. In some embodiments, the NRI, such as atomoxetine or a pharmaceutically acceptable salt thereof, is present in an amount of from about 25 to about 100 mg. In some embodiments, the CAI, such as acetazolamide, is present in an amount of from about 150 mg to about 750 mg. In some embodiments, the carbonic anhydrase inhibitor, such as acetazolamide, is present in an amount of about 500 mg. In some embodiments, the carbonic anhydrase inhibitor, such as acetazolamide, is present in an amount of about 250 mg. In some embodiments, the NRI and CAI are formulated as separate compositions. In some embodiments, the NRI and CAI are formulated in a single composition. In some embodiments, the separate compositions or single composition are an oral administration form. In some embodiments, the oral administration form is a syrup, pill, tablet, troche, capsule, or patch. In some embodiments, the pharmaceutical composition is for use in treating a subject having a condition associated with central hypoventilation. In some embodiments, the condition associated with central hypoventilation is obesity hypoventilation syndrome (OHS) or obesity-related sleep hypoventilation (ORSH). In some embodiments, the condition associated with central hypoventilation is obesity hypoventilation syndrome (OHS). In some embodiments, the condition associated with central hypoventilation is obesity-related sleep hypoventilation (ORSH). In some embodiments, the pharmaceutical composition is administered daily. In some embodiments, the pharmaceutical composition is administered twice-daily.

Also provided herein is a norepinephrine reuptake inhibitor (NRI) and a carbonic anhydrase inhibitor (CAI), for use in treating a subject having a condition associated with central hypoventilation.

Further provided herein is a therapeutic combination of (i) a norepinephrine reuptake inhibitor (NRI) and (ii) a carbonic anhydrase inhibitor (CAI), for use in treating a subject having a condition associated with central hypoventilation.

Unless otherwise defined, all technical and scientific terms used herein have the same meaning as commonly understood by one of ordinary skill in the art to which this invention belongs. Methods and materials are described herein for use in the present invention; other suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and from the claims.

### DETAILED DESCRIPTION

Obese subjects have an increased ventilator demand and an elevated work of breathing, in addition to slight respiratory muscle weakness and diminished respiratory compliance. Obese individuals generally have an increased central respiratory drive compared with normal weight patients to compensate for the increased ventilatory requirements. Despite this, in 20% of obese subjects (BMI>30 kg/m²) evaluated in sleep clinics, OSA is associated with diurnal hypercapnia (PaCO₂>45 mm Hg), which defines the obesity-hypoventilation syndrome (OHS) (1).

Compared to isolated OSA, OHS is characterized by increased morbidity and mortality due to cardiovascular and metabolic diseases associated with systemic inflammation, endothelial dysfunction and insulin resistance and the diagnosis is often made only after acute respiratory failure (2). However, OHS remains largely undiagnosed and untreated until patients require intensive care unit admission for acute decompensation (2, 3).

Lack in ventilatory responsiveness to gas alterations and insufficient ability to compensate to upper airway obstruction are pathophysiological implications of OHS. As the balance between obesity-related respiratory charge and ventilatory responsiveness to gas alterations may be conserved during daytime, it might be lost during sleep, determining initially an isolated nocturnal hypoventilation. Therefore, hypoventilation is most and primarily pronounced during sleep as a consequence of sleep-related physiological adaptations, which may or may not be also associated to sleep apnea. Indeed, it recently has been stated that isolated sleep hypoventilation (defined as a transcutaneous carbon dioxide pressure, PtcCO₂>55 mmHg or >50 mmHg if PtcCO₂ increases by more than 10 mmHg for more than 10 minutes of sleep compared to awake supine value) without awake hypercapnia precedes OHS, similar to what is observed in neuromuscular and chest wall diseases (4). This condition is called obesity-related sleep hypoventilation (ORSH) and it is now considered an early stage of hypoventilation in patients with obesity(5, 6). Recent evidence has shown that around 20% of patients with grade III obesity present ORSH (7, 8), thus supporting a massive underestimation of the disease.

The only available therapeutic option for OHS is, up to now, to address the underlying pathophysiological condition, such as the upper airways obstruction by positive air pressure (PAP). However, the adherence to treatment is often very difficult with low tolerance and consequent reduced compliance to PAP, resulting in under-treatment of OHS.

Acetazolamide is a diuretic that inhibits carbonic anhydrase, increases HCO₃ excretion, and causes metabolic acidosis thus stimulating ventilation. Addressing the plant gain, acetazolamide acts as a mild ventilatory stimulant and stabilizes breathing, thus reducing shallow respirations followed by hyperventilations typical of ORSH patients. Acetazolamide has been shown to improve alveolar ventilation in patients with OHS (9, 10). However, acetazolamide alone does not affect all of the pathogenic contributors to obesity hypoventilation.

No pharmacologic treatments of OHS or ORSH have been approved to date.

### Methods of Treatment

The methods described herein include methods for the treatment of conditions associated with central hypoventilation. In some embodiments, the condition is obesity hypoventilation syndrome (OHS) or obesity-related sleep hypoventilation (ORSH).

Generally, the methods include administering an effective amount of (i) a norepinephrine reuptake inhibitor (NRI) and (ii) a carbonic anhydrase inhibitor to a subject who is in need of, or who has been determined to be in need of, such treatment. In certain embodiments the methods include administering an effective amount of (i) atomoxetine or a pharmaceutically acceptable salt thereof and (ii) acetazolamide or a pharmaceutically acceptable salt thereof to a subject who is in need of, or who has been determined to be in need of, such treatment.

In some embodiments, the methods further comprise administering a therapeutically effective amount of (iii) a hypnotic. In certain embodiments, the methods include administering an effective amount of (i) atomoxetine or a pharmaceutically acceptable salt thereof, (ii) acetazolamide or a pharmaceutically acceptable salt thereof, and (iii) trazodone or a pharmaceutically acceptable salt thereof to a subject who is in need of, or who has been determined to be in need of, such treatment.

In some embodiments, the methods further comprise administering a therapeutically effective amount of (iii) an antimuscarinic agent. In certain embodiments, the methods include administering an effective amount of (i) atomoxetine or a pharmaceutically acceptable salt thereof, (ii) acetazolamide or a pharmaceutically acceptable salt thereof, and (iii) oxybutynin or a pharmaceutically acceptable salt thereof to a subject who is in need of, or who has been determined to be in need of, such treatment. In certain embodiments, the methods include administering an effective amount of (i) atomoxetine or a pharmaceutically acceptable salt thereof, (ii) acetazolamide or a pharmaceutically acceptable salt thereof, and (iii) (R)-oxybutynin or a pharmaceutically acceptable salt thereof to a subject who is in need of, or who has been determined to be in need of, such treatment.

As used in this context, to “treat” means to ameliorate at least one symptom of the disorder associated with central hypoventilation. In some embodiments, a disorder associated with central hypoventilation is obesity hypoventilation syndrome (OHS). Often, OHS, results in sleepiness, lack of energy, breathlessness, headache, and depression during the daytime. At nighttime, OHS results in loud and frequent snoring during sleep and/or breathing pauses. OHS patients can also have right heart failure with lower extremity edema. Thus, a treatment can result in reduction of snoring, apneas, breathing pauses, breathlessness, headache, and other symptoms associated with OHS.

In some embodiments, a disorder associate with central hypoventilation is obesity-related sleep hypoventilation (ORSH). ORSH can be described as a condition having isolated sleep hypoventilation (defined as a transcutaneous carbon dioxide pressure, PtcCO₂>55 mmHg or >50 mmHg if PtcCO₂ increases by more than 10 mmHg for more than 10 minutes of sleep compared to awake supine value) without awake hypercapnia, which precedes OHS, similar to what is observed in neuromuscular and chest wall diseases.

In general, an “effective amount” of a compound refers to an amount sufficient to elicit the desired biological response, e.g., to treat a condition associated with central hypoventilation e.g., to treat obesity hypoventilation syndrome (OHS) or obesity-related sleep hypoventilation (ORSH).

Patients with a “hypoventilation syndromes” generally have mild hypercarbia or elevated serum bicarbonate levels when awake, which sometimes worsen during sleep. Hypoventilation syndromes include, and are not limited to, obesity hypoventilation syndrome (OHS).

“Hypoventilation” is defined as elevated levels of arterial carbon dioxide (pCO₂), e.g., elevated by at least 10 mm Hg above the upper limit of normal. Treatment of hypoventilation syndromes is generally aimed at correcting or improving the waking pCO₂.

An effective amount can be administered in one or more administrations, applications or dosages. The compositions can be administered from one or more times per day to one or more times per week; including once every other day. In some embodiments, the compositions are administered daily. The skilled artisan will appreciate that certain factors may influence the dosage and timing required to effectively treat a subject, including but not limited to the severity of the disease or disorder, previous treatments, the general health and/or age of the subject, and other diseases present. Moreover, treatment of a subject with a therapeutically effective amount of the therapeutic compounds described herein can include a single treatment or a series of treatments.

As used herein, and unless otherwise specified, a “therapeutically effective amount” of a compound is an amount sufficient to provide a therapeutic benefit in the treatment of a disease, disorder or condition, or to delay or minimize one or more symptoms associated with the disease, disorder or condition. A therapeutically effective amount of a compound means an amount of therapeutic agent, alone or in combination with other therapies, which provides a therapeutic benefit in the treatment of the disease, disorder or condition. The term “therapeutically effective amount” can encompass an amount that improves overall therapy, reduces or avoids symptoms or causes of disease or condition, or enhances the therapeutic efficacy of another therapeutic agent. In some embodiments, the therapeutically effective amount encompasses an amount that normalizes or improves waking pCO₂ levels.

As used herein, the terms “subject” and “patient” are used interchangeably. The terms “subject” and “patient” refer to an animal (e.g., a bird such as a chicken, quail or turkey, or a mammal), specifically a “mammal” including a non-primate (e.g., a cow, pig, horse, sheep, rabbit, guinea pig, rat, cat, dog, and mouse) and a primate (e.g., a monkey, chimpanzee and a human), and more specifically a human. In one embodiment, the subject is a non-human animal such as a farm animal (e.g., a horse, cow, pig or sheep), or a pet (e.g., a dog, cat, guinea pig or rabbit). In a preferred embodiment, the subject is a human.

As used herein, “pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable salts” includes “pharmaceutically acceptable acid addition salts” and “pharmaceutically acceptable base addition salts.” “Pharmaceutically acceptable acid addition salts” refers to those salts that retain the biological effectiveness of the free bases and that are not biologically or otherwise undesirable, formed with inorganic acids such as hydrochloric acid, hydrobromic acid, sulfuric acid, nitric acid, phosphoric acid, and the like, as well as organic acids such as acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

“Pharmaceutically acceptable base addition salts” include those derived from inorganic bases such as sodium, potassium, lithium, ammonium, calcium, magnesium, iron, zinc, copper, manganese, aluminum salts, and the like. Exemplary salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines, and basic ion exchange resins, such as isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine, N-ethylpiperidine, polyamine resins, and the like. Exemplary organic bases are isopropylamine, diethylamine, ethanolamine, trimethylamine, dicyclohexylamine, choline, and caffeine. (See, for example, S. M. Berge, et al., “Pharmaceutical Salts,” J. Pharm. Sci., 1977; 66:1-19 which is incorporated herein by reference.)

As used herein, the term “unit dosage form” is defined to refer to the form in which the compound is administered to a subject. Specifically, the unit dosage form can be, for example, a pill, capsule, or tablet. In some embodiments, the unit dosage form is a capsule.

As used herein, “solid dosage form” means a pharmaceutical dose(s) in solid form, e.g., tablets, capsules, granules, powders, sachets, reconstitutable powders, dry powder inhalers and chewables.

For the compounds disclosed herein, single stereochemical isomers, as well as enantiomers, diastereomers, cis/trans conformation isomers, and rotational isomers, and racemic and non-racemic mixtures thereof, are within the scope of the invention. Unless otherwise indicated, all tautomeric forms of the compounds disclosed herein are within the scope of the invention.

Atomoxetine is the generic name of the pharmaceutical substance with the chemical name (−)-N-Methyl-3-phenyl-3-(o-tolyloxy)-propylamine, and its pharmaceutical salts. Atomoxetine is the R(−)-isomer as determined by x-ray diffraction. In some embodiments, atomoxetine may be atomoxetine hydrochloride.

Acetazolamide is the generic name of the pharmaceutical substance with the chemical name N-(5-Sulfamoyl-1,3,4-thiadiazol-2-yl)acetamide, and its pharmaceutical salts. Acetazolamide is available as a generic medication as well as sold under the trade names Diamox, Dacarb, and others.

In some embodiments, the methods include administering a dose of from about 20 mg to about 200 mg of atomoxetine or a pharmaceutically acceptable salt thereof (or a dose equivalent of another NRI). In some embodiments, the dose of atomoxetine or a pharmaceutically acceptable salt thereof is from about 25 mg to about 100 mg. In some embodiments, the dose of atomoxetine or pharmaceutically acceptable salt thereof is from about 40 mg to about 80 mg. In some embodiments, the dose of atomoxetine or pharmaceutically acceptable salt thereof is from about 20 mg to about 50 mg. In some embodiments, the dose of atomoxetine or a pharmaceutically acceptable salt thereof is from about 50 mg to about 100 mg. In some embodiments, the dose of atomoxetine or pharmaceutically acceptable salt thereof is about 25 mg. In some embodiments, the dose of atomoxetine or pharmaceutically acceptable salt thereof is about 40 mg. In some embodiments, the dose of atomoxetine or pharmaceutically acceptable salt thereof is about 50 mg. In some embodiments, the dose of atomoxetine or pharmaceutically acceptable salt thereof is about 80 mg. In some embodiments, the dose of atomoxetine or pharmaceutically acceptable salt thereof is about 100 mg. In some embodiments, the dose is a daily dose, i.e., is administered once per day. In some embodiments, the dose is a twice-daily dose, i.e., is administered in two separate administrations per day, e.g., once in the morning and once at bedtime. In some embodiments, the dose of atomoxetine or pharmaceutically acceptable salt thereof is a daily dose of about 25 mg. In some embodiments, the dose of atomoxetine or pharmaceutically acceptable salt thereof is a daily dose of about 50 mg. In some embodiments, the dose of atomoxetine or pharmaceutically acceptable salt thereof is a daily dose of about 100 mg. In some embodiments, the dose of atomoxetine or pharmaceutically acceptable salt thereof is a total daily dose of 25 mg, administered in two separate administrations. In some embodiments, the dose of atomoxetine or pharmaceutically acceptable salt thereof is a total daily dose of about 50 mg, administered in two separate administrations. In some embodiments, the dose of atomoxetine or pharmaceutically acceptable salt thereof is a total daily dose of about 100 mg, administered in two separate administrations. In some embodiments, the two separate administrations are a morning administration and an administration at bedtime. In some embodiments, the two separate administrations are a morning administration and an evening administration.

In some embodiments, the methods include administering a dose of from about 50 mg to about 1000 mg acetazolamide (or a dose equivalent thereof of another CAI), from about 100 mg to about 800 mg acetazolamide, from about 150 mg to about 750 mg acetazolamide, from about 250 mg to about 750 mg, from about 500 mg to about 750 mg acetazolamide, or from about 450 mg to about 650 mg acetazolamide. In some embodiments, the dose of acetazolamide is about 250 mg. In some embodiments, the dose of acetazolamide is about 500 mg. In some embodiments, the dose is a daily dose, i.e., is administered once per day. In some embodiments, the dose is a twice-daily dose, i.e., is administered in two separate administrations per day, e.g., once in the morning and once at bedtime. In some embodiments, the dose of acetazolamide is a daily dose of about 250 mg. In some embodiments, the dose of acetazolamide is a daily dose of about 500 mg. In some embodiments, the dose of acetazolamide is a total daily dose of about 250 mg, administered in two separate administrations. In some embodiments, the dose of acetazolamide is a total daily dose of about 500 mg, administered in two separate administrations. In some embodiments, the two separate administrations are a morning administration and an administration at bedtime. In some embodiments, the two separate administrations are a morning administration and an evening administration.

In some embodiments, the NRI and CAI are administered in the absence of an antimuscarinic therapy. In some embodiments, the NRI and CAI are administered in the absence of other active agents.

### Pharmaceutical Compositions

Also provided herein are pharmaceutical compositions comprising (i) a norepinephrine reuptake inhibitor (NRI) and (ii) a carbonic anhydrase inhibitor (CAI), as active ingredients. The active ingredients can be in a single composition or in separate compositions. In certain embodiments, the pharmaceutical compositions include (i) atomoxetine or a pharmaceutically acceptable salt thereof and (ii) acetazolamide or a pharmaceutically acceptable salt thereof, as active ingredients. In some embodiments, the pharmaceutical composition does not comprise an antimuscarinic agent. In some embodiments, the NRI and CAI are the sole active ingredients in the pharmaceutical composition.

Exemplary norepinephrine reuptake inhibitors (NRIs) include the selective NRIs, e.g., amedalin (UK-3540-1), atomoxetine (Strattera), CP-39,332, daledalin (UK-3557-15), edivoxetine (LY-2216684), esreboxetine, lortalamine (LM-1404), nisoxetine (LY-94,939), reboxetine (Edronax, Vestra), talopram (Lu 3-010), talsupram (Lu 5-005), tandamine (AY-23,946), viloxazine (Vivalan); and the non-selective NRIs, e.g., amitriptiline, amoxapine, bupropion, ciclazindol, desipramine, desvenlafaxine, dexmethilphenidate, diethylpropion, doxepin, duloxetine, imipramine, levomilnacipran, manifaxine (GW-320,659), maprotiline, methylphenidate, milnacipran, nefazodone, nortriptyline, phendimetrazine, phenmetrazine, protryptyline, radafaxine (GW-353,162), tapentadol (Nucynta), teniloxazine (Lucelan, Metatone) and venlafaxine; and pharmaceutically acceptable salts thereof.

In some embodiments, the NRI is atomoxetine or a pharmaceutically acceptable salt thereof. In some embodiments, the NRI is reboxetine or a pharmaceutically acceptable salt thereof.

Exemplary carbonic anhydrase inhibitor (CAI) include acetazolamide, dichlorophenamide, dorzolamide, brinzolamide, methazolamide, zonisamide, ethoxzolamide, topiramate, sultiame, and any combinations thereof, including pharmaceutically acceptable salts thereof.

In some embodiments, the carbonic anhydrase inhibitor is acetazolamide or a pharmaceutically acceptable salt thereof.

Pharmaceutical compositions typically include a pharmaceutically acceptable carrier. As used herein the language “pharmaceutically acceptable carrier” includes saline, solvents, dispersion media, diluents, coatings, antibacterial and antifungal agents, isotonic and absorption delaying agents, and the like, compatible with pharmaceutical administration.

Supplementary active compounds can also be incorporated into the compositions, e.g., muscarinic receptor antagonists (MRAs), e.g., oxybutynin, or a pharmaceutically acceptable salt thereof or a hypnotic. In some embodiments, the pharmaceutical composition further comprises a muscarinic receptor antagonists (MRAs). Exemplary muscarinic receptor antagonists (MRAs) include atropine, propantheline, bethanechol, solifenacin, darifenacin, tolterodine, fesoterodine, trospium, and oxybutynin, and pharmaceutically acceptable salts thereof, which have activity on the M2 receptor. Other exemplary antimuscarinics include anisotropine, benztropine, biperiden, clidinium, cycrimine, dicyclomine, diphemanil, diphenidol, ethopropazine, glycopyrrolate, hexocyclium, isopropamide, mepenzolate, methixe ne, methscopolamine, oxyphencyclimine, oxyphenonium, procyclidine, scopolamine, tridihexethyl, and trihexyphenidyl, and pharmaceutically acceptable salts thereof.

In some embodiments, the muscarinic receptor antagonist is oxybutynin or (R)-oxybutynin, or a pharmaceutically acceptable salt thereof. As used herein, (R)-oxybutynin refers to the (R)-oxybutynin stereoisomer substantially free of other stereoisomers of oxybutynin.

In some embodiments, the pharmaceutical composition further comprises a hypnotic. Exemplary hypnotics include zolpidem, zopiclone, eszopiclone, trazodone, zaleplon, benzodiazepines, gabapentin, tiagabine, and xyrem or pharmaceutically acceptable salts thereof. In some embodiments, the hypnotic is trazodone or a pharmaceutically acceptable salt thereof.

Pharmaceutical compositions are typically formulated to be compatible with its intended route of administration. Examples of routes of administration include systemic oral, transdermal administration, and parenteral administration.

Methods of formulating suitable pharmaceutical compositions using pharmaceutically acceptable carriers are known in the art, see, e.g., Remington: The Science and Practice of Pharmacy, 21st ed., 2005; and the books in the series Drugs and the Pharmaceutical Sciences: a Series of Textbooks and Monographs (Dekker, NY). For example, oral compositions generally include an inert diluent or an edible carrier. For the purpose of oral therapeutic administration, the active compound(s) can be incorporated with excipients and used in the form of pills, tablets, troches, or capsules, e.g., gelatin capsules. Oral compositions can also be prepared using a fluid carrier. In some embodiments, a composition according to the present invention may be a unit dosage form. In some embodiments, a composition according to the present invention may be a solid dosage form, e.g., a tablet or capsule.

Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

Systemic administration of the compounds as described herein can also be by transdermal means, e.g., using a patch, gel, or lotion, to be applied to the skin. For transdermal administration, penetrants appropriate to the permeation of the epidermal barrier can be used in the formulation. Such penetrants are generally known in the art. For example, for transdermal administration, the active compounds can formulated into ointments, salves, gels, or creams as generally known in the art. The gel and/or lotion can be provided in individual sachets, or via a metered-dose pump that is applied daily; see, e.g., Cohn et al., Ther Adv Urol. 2016 April; 8(2): 83-90.

In one embodiment, the therapeutic compounds are prepared with carriers that will protect the therapeutic compounds against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Such formulations can be prepared using standard techniques, or obtained commercially, e.g., from Alza Corporation and Nova Pharmaceuticals, Inc. Liposomal suspensions can also be used as pharmaceutically acceptable carriers. These can be prepared according to methods known to those skilled in the art, for example, as described in U.S. Pat. No. 4,522,811.

The pharmaceutical compositions can be included in a container, pack, or dispenser together with instructions for administration or use in a method described herein.

The pharmaceutical compositions can be included in a container, pack, or dispenser together with instructions for administration or use in a method described herein.

In some embodiments, the pharmaceutical composition is for use in treating a condition associated with central hypoventilation. In some embodiments, the condition is obesity hypoventilation syndrome (OHS). In some embodiments, the condition associated with central hypoventilation is obesity-related sleep hypoventilation (ORSH). In some embodiments, the condition is obesity-related sleep hypoventilation (ORSH).

### Combinations

Also provided herein is a norepinephrine reuptake inhibitor (NRI) and a carbonic anhydrase inhibitor (CAI), for use in treating a subject having a condition associated with central hypoventilation. Further provided herein is a therapeutic combination of (i) a norepinephrine reuptake inhibitor (NRI) and (ii) a carbonic anhydrase inhibitor (CAI), for use in treating a subject having a condition associated with central hypoventilation. In some embodiments, the condition is obesity hypoventilation syndrome (OHS). In some embodiments, the condition associated with central hypoventilation is obesity-related sleep hypoventilation (ORSH).

### EXAMPLES

The invention is further described in the following examples, which do not limit the scope of the invention described in the claims.

### Example 1. Pharmacological Therapy for Obesity Hypoventilation Syndrome

### Introduction

Obese subjects have an increased ventilator demand and an elevated work of breathing, in addition to slight respiratory muscle weakness and diminished respiratory compliance. Thus, beside the increased prevalence of obstructive sleep apnea (OSA) that grows proportionally with the body mass index (BMI), obese individuals, generally have also an increased central respiratory drive compared with normal weight patients to compensate for the increased ventilatory requirements. Despite this, in 20% of obese subjects (BMI>30 kg/m²) evaluated in sleep clinics, OSA is associated with diurnal hypercapnia (PaCO₂>45 mm Hg), which defines the obesity-hypoventilation syndrome (OHS) (1).

Compared to isolated OSA, OHS are characterized by increased morbidity and mortality due to cardiovascular and metabolic diseases associated with systemic inflammation, endothelial dysfunction and insulin resistance and the diagnosis is often made only after acute respiratory failure (2). However, OHS remains largely undiagnosed and untreated until patients require intensive care unit admission for acute decompensation (2, 3). Therefore, a timely identification and management of the earlier OHS stages might improve patients' prognosis.

Lack in ventilatory responsiveness to gas alterations and insufficient ability to compensate to upper airway obstruction are the pathophysiological implications of OHS. As the balance between obesity-related respiratory charge and ventilatory responsiveness to gas alterations may be conserved daytime, it might be lost during sleep, determining initially an isolated nocturnal hypoventilation. Therefore, hypoventilation is most and primarily pronounced during sleep as a consequence of sleep-related physiological adaptations, that might be or not also associated to sleep apnea. Indeed, recently it has been stated that isolated sleep hypoventilation (defined as a transcutaneous carbon dioxide pressure, PtcCO₂>55 mmHg or >50 mmHg if PtcCO₂ increases by more than 10 mmHg for more than 10 minutes of sleep compared to awake supine value) without awake hypercapnia precedes OHS, similarly to what is observed in neuromuscular and chest wall diseases (4). This condition is called obesity-related sleep hypoventilation (ORSH) and it is now considered an early stage of hypoventilation in patients with obesity(5, 6). Recent evidence has shown that around 20% of patients with grade III obesity present ORSH (7, 8), thus supporting a massive underestimation of the disease.

The only available therapeutic option for OHS is, up to now, to address the underlying pathophysiological condition, such as the upper airways obstruction by positive air pressure (PAP). However, the adherence to treatment is often very difficult with low tolerance and consequent reduced compliance to PAP, resulting in under-treatment of OHS.

Acetazolamide is a diuretic that inhibits carbonic anhydrase, increases HCO₃ excretion, and causes metabolic acidosis thus stimulating ventilation. Addressing the plant gain, acetazolamide acts as a mild ventilatory stimulant and stabilizes breathing, thus reducing the shallow respirations followed by hyperventilations typical of ORSH patients. Previously, acetazolamide has been shown to improve alveolar ventilation in patients with OHS (9, 10). However, acetazolamide alone does not affect all of the pathogenic contributors to obesity hypoventilation. We decided to explore atomoxetine in combination with acetazolamide. Atomoxetine is a noradrenergic drug which reactivates the upper airway muscle during sleep reducing the collapsibility(11).

Our aim will be to test the efficacy of the combination of atomoxetine and acetazolamide in adults with obesity hypoventilation.

### Study Design

This will be randomized, double blind, placebo-controlled, cross-over, single center efficacy study of the combination of atomoxetine and acetazolamide in adults with ORSH.

12 participants will be randomized equally to receive the combinations of atomoxetine 80 mg and acetazolamide 500 mg, or matching placebo. Dosing of the study treatment will occur immediately prior to bedtime.

Among the 12 subjects with ORSH (early stage of OHS), we estimate that at least 2 of them will also present diurnal hypercapnia (advanced OHS).

Study participants will undergo eligibility screening that may include a 1-night inpatient PSG test with PtcCO₂ monitoring. Blood gas analyses will also be performed in the morning.

After 1 week, on the final night of dosing, participants will return to the study site for inpatient PSG. Participants will have 7-10 days of washout and will switch to the other arm of the study. After 5 days, on the final night of dosing, participants will return to the study site for inpatient PSG.

The primary efficacy endpoint is the mean nocturnal PtCO₂ from screening/baseline to final night of treatment with study treatment.

Secondary outcomes will include apnea\hypopnea index (AHI) and nocturnal saturation (hypoxic burden and mean SpO2).

### Inclusion Criteria

Male or female participants between 18 to 70 years of age

BMI>35 kg/m², inclusive, at the pre-PSG visit

Nocturnal hypoventilation defined as PtcCO₂>55 mmHg or >50 mmHg if PtcCO₂ increases by more than 10 mmHg for more than 10 minutes of sleep compared to awake supine value

Previous surgical treatment for OSA is allowed if >1 year prior to enrollment.

Exclusion criteria

History of narcolepsy.

Clinically significant craniofacial malformation.

Clinically significant cardiac or lung disease (heart failure, COPD, ILD) disease or hypertension requiring more than 3 medications for control.

History of schizophrenia, schizoaffective disorder or bipolar disorder according to Diagnostic and Statistical Manual of Mental Disorders-V (DSM-V) or International Classification of Disease tenth edition criteria.

History of attempted suicide or suicidal ideation within 1 year prior to screening, or current suicidal ideation.

Positive screen for drugs of abuse or substance use disorder as defined in DSM-V within 12 months prior to Screening Visit.

A significant illness or infection requiring medical treatment in the past 30 days.

Clinically significant cognitive dysfunction.

Untreated narrow angle glaucoma.

Women who are pregnant or nursing.

History of oxygen therapy

Treatment with strong cytochrome P450 3A4 (CYP3A4) inhibitors, or monoamine oxidase inhibitors (MAOI) or linezolid within 14 days of the start of treatment, or concomitant with treatment.

Central apnea index>5/hour

### Interventions

There will be 2 treatment groups, as follows:

| Group | Treatment | Subjects (n) |
|---|---|---|
| 1 | Atomoxetine 80 mg/Acetazolamide 500 mg first, | 6 |
| | placebo second | |
| 2 | Placebo first, Atomoxetine 80 mg/Acetazolamide | 6 |
| | 500 mg second | |

### REFERENCES

1. Macavei V M, Spurling K J, Loft J, Makker H K. Diagnostic predictors of obesity-hypoventilation syndrome in patients suspected of having sleep disordered breathing. J Clin Sleep Med 2013; 9: 879-884.
2. Nowbar S, Burkart K M, Gonzales R, Fedorowicz A, Gozansky W S, Gaudio J C, Taylor M R, Zwillich C W. Obesity-associated hypoventilation in hospitalized patients: prevalence, effects, and outcome. Am J Med 2004; 116: 1-7.
3. Mokhlesi B. Obesity hypoventilation syndrome: a state-of-the-art review. Respir Care 2010; 55: 1347-1362; discussion 1363-1345.
4. Piper A J, Grunstein R R. Obesity hypoventilation syndrome: mechanisms and management. Am J Respir Crit Care Med 2011; 183: 292-298.
5. Randerath W, Verbraecken J, Andreas S, Arzt M, Bloch K E, Brack T, Buyse B, De Backer W, Eckert D J, Grote L, Hagmeyer L, Hedner J, Jennum P, La Rovere M T, Miltz C, McNicholas W T, Montserrat J, Naughton M, Pepin J L, Pevernagie D, Sanner B, Testelmans D, Tonia T, Vrijsen B, Wijkstra P, Levy P. Definition, discrimination, diagnosis and treatment of central breathing disturbances during sleep. Eur Respir J 2017; 49.
6. Randerath W J, BaHammam A S. Overlooking Obesity Hypoventilation Syndrome: The Need for Obesity Hypoventilation Syndrome Staging and Risk Stratification. Annals of the American Thoracic Society 2020; 17: 1211-1212.
7. Sivam S, Yee B, Wong K, Wang D, Grunstein R, Piper A. Obesity Hypoventilation Syndrome: Early Detection of Nocturnal-Only Hypercapnia in an Obese Population. J Clin Sleep Med 2018; 14: 1477-1484.
8. Perger E, Aron-Wisnewsky J, Arnulf I, Oppert J M, Redolfi S. Diagnostic approach to sleep disordered-breathing among patients with grade III obesity. Sleep Med 2021; 82: 18-22.
9. Powers M A. Obesity hypoventilation syndrome: bicarbonate concentration and acetazolamide. Respir Care 2010; 55: 1504-1505.
10. Raurich J M, Rialp G, Ibáñez J, Llompart-Pou J A, Ayestarin I. Hypercapnic respiratory failure in obesity-hypoventilation syndrome: CO₂ response and acetazolamide treatment effects. Respir Care 2010; 55: 1442-1448.
11. Taranto-Montemurro L, Messineo L, Sands S A, Azarbarzin A, Marques M, Edwards B A, Eckert D J, White D P, Wellman A. The Combination of Atomoxetine and Oxybutynin Greatly Reduces Obstructive Sleep Apnea Severity. A Randomized, Placebo-controlled, Double-Blind Crossover Trial. Am J Respir Crit Care Med 2019; 199: 1267-1276.

### Example 2. Crossover, Double-Blind, Phase 2 Study of a Fixed Dose Combination of Atomoxetine and Acetazolamide Versus Placebo in Obesity Hypoventilation Syndrome

This is a randomized, double blind, placebo-controlled, crossover study of the combination of atomoxetine 100 mg plus acetazolamide 500 mg (Ato/Actz) in adults with OHS documented by PSG with PtcCO2 monitoring. The dosage will be increased gradually: the administration of atomoxetine 50 mg and acetazolamide 500 mg for 7 days will be followed by the administration of atomoxetine 100 mg plus acetazolamide 500 mg for other 7 days to allow better treatment tolerance. Approximately 15 participants will be randomized to receive the combination of Ato/Actz or matching placebo. After 14 days of treatment and a washout period of 3-14 days, the patients will take the alternative treatment for 14 days. Alternatively, participants may undergo treatment periods of 30 or more days.

Study participants will undergo a screening visit prior to the conduct of any study-specific procedures to ascertain enrollment eligibility. Participants who otherwise meet all enrollment criteria will undergo a 1-night inpatient PSG test with PtcCO2 monitoring. Arterial blood gas tests will be performed if not available in the previous 6 months. For participants who are eligible and enroll in the study, the screening PSG night will serve as the baseline measure for PtcCO2 and other PSG efficacy and safety endpoints. Participants will also receive a pulse oximetry device to be worn nightly at home for all nights between PSG nights. On the final night of investigational product dosing, participants will return for a second inpatient PSG (Visit 1) with PtcCO2 and a blood gas test the morning after the PSG. After a washout period they will cross-over (Visit 2) to the other arm of the study and will continue to wear the pulse oximeter each night at home. After 14 days (or alternatively 30 or more days) of treatment they will undergo an inpatient PSG (Visit 3) with PtcCO2 and an arterial blood gas test the morning after the PSG.

The primary outcome is arterial PaCO2.

### Interventions

There will be 2 treatment groups, as follows:

| Study Treatment Name: | Atomoxetine | Acetazolamide |
|---|---|---|
| Dosage Formulation: | Capsule | Capsule |
| Dosage Level: | 25 mg BID (twice a day) for 7 | 250 mg BID (twice a day) |
| | days followed by 50 mg BID for | for 14 days |
| | 7 days | |
| Route of Administration: | Oral | Oral |
| Dosing Instructions: | 1-2 capsules in the morning and | 1 capsule in the morning |
| | QHS (each night at bedtime) | and QHS (each night at |
| | with at least 20 mL water | bedtime) with at least 20 |
| | | mL water |

The overall duration will be up to 9 weeks, as follows: Up to 28 days to conduct screening and baseline PSG; 14 days randomized at-home study treatment; In-lab PSG night with PtcCO2 monitoring; 3-16 days washout (up to 16 days if necessary, for scheduling); 14 days cross-over to the other treatment arm; and Final in-lab PSG night with PtcCO2 monitoring. Alternatively 30 day (or more) treatment periods may be used in place of 14 day treatment periods.

### Inclusion Criteria

1. Participant must be able to understand the nature of the study and must have the opportunity to have any questions answered. Participant voluntarily agrees to participate in this study and signs an Ethic Committee-approved informed consent prior to performing any of the Screening Visit procedures.
2. Male or female participants between 18 to 75 years of age
3. BMI>35 kg/m², inclusive, at the pre-PSG visit
4. Presence of nocturnal hypoventilation defined as mean PtcCO₂>55 mmHg or >50 mmHg if PtcCO₂ increases by more than 10 mmHg for more than 10 minutes of sleep compared to awake supine value
5. Previous surgical treatment for OSA is allowed if >1 year prior to enrollment.
6. Participants known for OHS and having treatment are eligible for screening/baseline PSG if they report CPAP or mandibular advancement device or positional therapy intolerance or poor compliance (compliance is defined as use of CPAP or other treatments for 4 hours per night for 70% of nights; per participant self-report); Participants who had been using CPAP at least 4 hours nightly for at least 70% of the nights are eligible for further screening and baseline PSG for this study only if CPAP or other treatments will not have been used for 2 weeks prior to the screening/baseline PSG for this study.

### Exclusion Criteria

1. History of narcolepsy.
2. Clinically significant craniofacial malformation.
3. Clinically significant respiratory (COPD, ILD) or cardiac (Heart Failure, Atrial fibrillation, established severe peripheral arterial disease) disease or hypertension requiring more than 3 medications for control.
4. History of schizophrenia, schizoaffective disorder or bipolar disorder according to Diagnostic and Statistical Manual of Mental Disorders-V (DSM-V) or International Classification of Disease tenth edition criteria.
5. History of attempted suicide or suicidal ideation within 1 year prior to screening, or current suicidal ideation.
6. Positive screen for drugs of abuse or substance use disorder as defined in DSM-V within 12 months prior to Screening Visit.
7. A significant illness or infection requiring medical treatment in the past 30 days.
8. Clinically significant cognitive dysfunction or serious neurological disorder, including epilepsy/convulsions
9. Untreated narrow angle glaucoma.
10. Women who are pregnant or nursing.
11. History of oxygen therapy
12. Treatment with strong cytochrome P450 3A4 (CYP3A4) inhibitors, or monoamine oxidase inhibitors (MAOI) or linezolid within 14 days of the start of treatment, or concomitant with treatment.
13. History of pheochromocytoma
14. History of diabetes with unstable glucose control in the past 15 days
15. Chronic use of more than 500 mg/day of Aspirin, due to the potential for an interaction of acetazolamide and very high doses of Aspirin (acetylsalicylic acid, a salicylate drug)
16. Allergies to sulfonamides—e.g. hydrochlorothiazide, furosemide, sulfasalazine, celecoxib, sumatriptan, and zonisamide.
17. History of Adrenocortical insufficiency
18. History of low sodium or potassium or evidence of low sodium or potassium at blood tests in the last year (if available)
19. History of hyperchloremic acidosis
20. Any condition that in the investigator's opinion would present an unreasonable risk to the participant, or which would interfere with their participation in the study or confound study interpretation.
21. Participant considered by the investigator, for any reason, an unsuitable candidate to receive Ato/Acz treatment or unable or unlikely to understand or comply with the dosing schedule or study evaluations.
22. History of using oral or nasal devices for the treatment of OSA may enroll as long as the devices are not used during participation in the study for at least 2 weeks prior to study begin.
23. History of using devices to affect participant sleeping position for the treatment of OSA, e.g. to discourage supine sleeping position, may enroll as long as the devices are not used during participation in the study.
24. Use of another investigational agent within 30 days or 5 half-lives, whichever is longer, prior to dosing.
25. Use of medications from the list of disallowed concomitant medications shown below.

### Atomoxetine Contraindication

Atomoxetine is contraindicated in narrow-angle glaucoma, patients concomitantly on MAOIs and those hypersensitive to atomoxetine or any of its excipients. Atomoxetine is also contraindicated in patients with current or history of pheochromocytoma, severe cardiac or vascular disorders in which the condition would be expected to deteriorate with clinically important increases in blood pressure (15-20 mmHg) or heart rate (20 bpm)

### Acetazolamide Contraindication

Acetazolamide is contraindicated in patients with marked hepatic disease or insufficiency; decreased sodium and/or potassium levels; adrenocortical insufficiency; cirrhosis; hyperchloremic acidosis; severe renal disease or dysfunction.

### Concomitant Therapy

Concomitant therapy with the following medications is disallowed:
MAOIs or other drugs that affect monoamine concentrations (e.g., rasagiline) [MAOIs are contraindicated for use with reboxetine]
Serotonin and Norepinephrine Reuptake Inhibitors (e.g., duloxetine, venlafaxine, mirtazapine)
Norepinephrine Reuptake Inhibitors (e.g., reboxetine)
Lithium
Tricyclic antidepressants (e.g., desipramine, imipramine)
Strong CYP2D6inhibitors (e.g., fluoxetine, paroxetine, quinidines, terbinafine) and other strong inhibitors cytochrome P450 (ketoconazole)
Drugs which determines electrolytic alterations (thiazides diuretics)
Benzodiazepines
Opioids
Drugs with clinically significant cardiac QT-interval prolonging effects (moxifloxacin, methadone, mefloquine,
Drugs known to lower seizure threshold (e.g., chloroquine, phenothiazine, butyrophenones, mefloquine, bupropion, tramadol)
Amphetamines
Antiepileptics
Modafinil or armodafinil
Beta2 agonists, (e.g., albuterol) if used more than 3 times/week
Antipsychotics
Pseudoephedrine, phenylephrine, oxymetazoline
Most drugs for Parkinson's, Alzheimer's (memantine), Huntington's, Amyotrophic Lateral Sclerosis, or drugs for other neurodegenerative diseases
Chronic use of more than 500 mg/day of Aspirin or salicylates, due to the potential for an interaction of acetazolamide and very high doses of Aspirin (acetylsalicylic acid, a salicylate drug)
Sodium Phosphates: diuretics may enhance the nephrotoxic effect of Sodium Phosphates. Specifically, the risk of acute phosphate nephropathy may be enhanced.

Medications that do not have substantial effects on the central nervous system (CNS), respiration, or muscle activity are generally allowed according to Investigator's opinion, if dose and frequency is stable for 3 months prior to enrollment and during the course of the study, including, but not necessarily limited to, the following drugs and drug classes:
Antihypertensives (angiotensin-converting-enzyme/angiotensin II receptor blocker inhibitors, calcium channel blockers, spironolactone, hydrochlorothiazide, etc.)
Statins
Alpha-1 antagonists (e.g., tamsulosin)
Chronic use of sedatives other than nonbenzodiazepine “Z-drugs” (zolpidem, zaleplon, eszopiclone)
Muscle relaxants
Antiemetics
Proton pump inhibitors and histamine h2 receptor blockers
Over-the-counter (OTC) antacids
Non-sedating antihistamines (e.g., cetirizine, loratadine)
Chronic use of Eszopiclone, zolpidem, or zaleplon
Melatonin
Non-steroidal anti-inflammatory drugs and acetaminophen
Laxatives
Erectile dysfunction drugs
Inhaled corticosteroids (e.g., fluticasone)
Antidiabetics
Ocular hypotensives and other ophthalmics (e.g., timolol)
Hormonal therapy (e.g., estrogen replacement or anti-estrogens) and hormonal
contraceptives
Thyroid medications
Anticoagulants
OTC topicals (e.g., topical pain relievers)
Osteoporosis drugs

### Outcomes

The following outcomes will be studied.

Primary Outcome: Change in mean nocturnal transcutaneous CO2 pressure (PtcCO2) on Ato/Actz vs placebo

### Secondary Outcomes

Proportion of participants without nocturnal hypoventilation on Ato/Actz vs placebo
Change in AHI on Ato/Actz vs placebo

### Exploratory Outcomes

Change in total time with Oxygen Saturation (SaO2)<90%
Change in mean SaO2
Change in minimum SaO2
Change in AHI4% and hypoxic burden
Change in PaCO2 during wakefulness at arterial blood gas analysis.
Change in ESS and SAQLI
Change in heart rate variability, assessed by electrocardiogram (EKG) during PSG
Change in Patient Global Impression of OSA Severity (PGI-S)
Change in Psychomotor Vigilance Test (PVT)

### Measurements of Outcomes

The Participant Global Impression of Severity (PGI-S) is a global index that may be used to rate the severity of a specific condition, (i.e., it is a single-state scale). The scale consists of a 1-item questionnaire designed to assess the participant's impression of disease severity. The scale is considered to have clinical relevance for the participant because it allows participants to respond based on factors that they judge to be the most important in their health status.

The Psychomotor Vigilance Test (PVT) is a sustained-attention, reaction-timed task that measures the speed with which subjects respond to a visual stimulus.

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of treating a subject having a condition associated with central hypoventilation, the method comprising administering to a subject in need thereof an effective amount of (i) a norepinephrine reuptake inhibitor (NRI) and (ii) a carbonic anhydrase inhibitor (CAI).

2. The method of claim 1, wherein the NRI is a norepinephrine selective reuptake inhibitor (NSRI).

3. The method of claim 2, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

4. The method of claim 1, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

5. The method of claim 1, wherein the NRI is reboxetine or a pharmaceutically acceptable salt thereof.

6. The method of claim 1, wherein the NRI is atomoxetine or a pharmaceutically acceptable salt thereof.

7. The method of any one of claims 1-6, wherein the CAI is selected from the group consisting of acetazolamide, dichlorophenamide, dorzolamide, brinzolamide, methazolamide, zonisamide, ethoxzolamide, topiramate, sultiame, and any combination thereof, or a pharmaceutically acceptable salt thereof.

8. The method of claim 7, wherein the CAI is acetazolamide or a pharmaceutically acceptable salt thereof.

9. The method of any one of claims 1-8, wherein the NRI, such as atomoxetine or a pharmaceutically acceptable salt thereof, is administered at a dosage of from about 20 to about 200 mg.

10. The method of claim 9, wherein the NRI, such as atomoxetine or a pharmaceutically acceptable salt thereof, is administered at a dosage of from about 25 to about 100 mg.

11. The method of any one of claims 1-10, wherein the CAI, such as acetazolamide, is administered at a dosage of from about 150 mg to about 750 mg.

12. The method of claim 11, wherein the carbonic anhydrase inhibitor, such as acetazolamide, is administered at a dosage of about 500 mg.

13. The method of claim 9 or 10, wherein the dosage of atomoxetine or a pharmaceutically acceptable salt thereof is administered in two separate administrations per day.

14. The method of claim 11 or 12, wherein the dosage of acetazolamide or a pharmaceutically acceptable salt thereof is administered in two separate administrations per day.

15. The method of claims 1-8, wherein the NRI is atomoxetine or a pharmaceutically acceptable salt thereof and is administered at a dosage of 25 to 100 mg, wherein the dosage of atomoxetine or a pharmaceutically acceptable salt thereof is administered in two separate administrations per day; and wherein the CAI is acetazolamide or a pharmaceutically acceptable salt thereof and is administered at a dosage of 250 to 750 mg, wherein the dosage of acetazolamide or a pharmaceutically acceptable salt thereof is administered in two separate administrations per day.

16. The method of any one of claims 1-15, wherein the NRI and CAI are administered as separate compositions.

17. The method of any one of claims 1-15, wherein the NRI and CAI are administered in a single composition.

18. The method of claim 16 or 17, wherein the separate compositions or single composition are an oral administration form.

19. The method of claim 18, wherein the oral administration form is a syrup, pill, tablet, troche, capsule, or patch.

20. The method of any one of claims 1-19, wherein the condition associated with central hypoventilation is obesity hypoventilation syndrome (OHS) or obesity-related sleep hypoventilation (ORSH).

21. The method of any one of claims 1-20, wherein the condition associated with central hypoventilation is obesity hypoventilation syndrome (OHS).

22. The method of any one of claims 1-20, wherein the condition associated with central hypoventilation is obesity-related sleep hypoventilation (ORSH).

23. A pharmaceutical composition comprising (i) a norepinephrine reuptake inhibitor (NRI) and (ii) a carbonic anhydrase inhibitor (CAI), and (iii) a pharmaceutically acceptable carrier, for use in treating a subject having a condition associated with central hypoventilation.

24. The pharmaceutical composition of claim 23, wherein the NRI is a norepinephrine selective reuptake inhibitor (NSRI).

25. The pharmaceutical composition of claim 24, wherein the NSRI is selected from the group consisting of amedalin, atomoxetine, CP-39,332, daledalin, edivoxetine, esreboxetine, lortalamine, nisoxetine, reboxetine, talopram, talsupram, tandamine, and viloxazine, or a pharmaceutically acceptable salt thereof.

26. The pharmaceutical composition of claim 23, wherein the NRI is a norepinephrine non-selective reuptake inhibitor (NNRI) selected from the group consisting of amitriptiline, amoxapine, bupropion, ciclazindol, desipramine, desvenlafaxine, dexmethilphenidate, diethylpropion, doxepin, duloxetine, imipramine, levomilnacipran, manifaxine, maprotiline, methylphenidate, milnacipran, nefazodone, nortriptyline, phendimetrazine, phenmetrazine, protryptyline, radafaxine, tapentadol, teniloxazine, and venlafaxine, or a pharmaceutically acceptable salt thereof.

27. The pharmaceutical composition of claim 23, wherein the NRI is reboxetine or a pharmaceutically acceptable salt thereof.

28. The pharmaceutical composition of claim 23, wherein the NRI is atomoxetine or a pharmaceutically acceptable salt thereof.

29. The pharmaceutical composition of claim 23, wherein the CAI is selected from the group consisting of acetazolamide, dichlorophenamide, dorzolamide, brinzolamide, methazolamide, zonisamide, ethoxzolamide, topiramate, sultiame, or a pharmaceutically acceptable salt thereof.

30. The pharmaceutical composition of claim 29, wherein the CAI is acetazolamide or a pharmaceutically acceptable salt thereof.

31. The pharmaceutical composition of any one of claims 23-30, wherein the NRI, such as atomoxetine or pharmaceutically acceptable salt thereof, is present in an amount of from about 20 to about 200 mg.

32. The pharmaceutical composition of claim 31, wherein the NRI, such as atomoxetine or pharmaceutically acceptable salt thereof, is present in an amount of from about 25 to about 100 mg.

33. The pharmaceutical composition of any one of claims 23-32, wherein the CAI, such as acetazolamide, is present in an amount of from about 150 mg to about 750 mg.

34. The pharmaceutical composition of claim 33, wherein the CAI, such as acetazolamide, is present in an amount of about 500 mg.

35. The pharmaceutical composition of claim 33, wherein the CAI, such as acetazolamide or a pharmaceutically acceptable salt thereof, is administered at a dosage of about 250 mg.

36. The pharmaceutical composition of any one of claims 23-35, wherein the NRI and CAI are formulated in separate compositions.

37. The pharmaceutical composition of any one of claims 23-35, wherein the NRI and CAI are formulated in a single composition.

38. The pharmaceutical composition of claims 36 or 37, wherein the separate composition or single composition are an oral administration form.

39. The pharmaceutical composition of claim 38, wherein the oral administration form is a syrup, pill, tablet, troche, capsule, or patch.

40. The pharmaceutical composition of any one of claims 23-39, wherein the condition associated with central hypoventilation is obesity hypoventilation syndrome (OHS) or obesity-related sleep hypoventilation (ORSH).

41. The pharmaceutical composition of claim 40, wherein the condition associated with central hypoventilation is obesity hypoventilation syndrome (OHS).

42. The pharmaceutical composition of claim 40, wherein the condition associated with central hypoventilation is obesity-related sleep hypoventilation (ORSH).

43. The pharmaceutical composition of any one of claims 23-42, wherein the pharmaceutical composition is administered daily.

44. The pharmaceutical composition of claim 43, wherein the pharmaceutical composition is administered twice-daily.

45. A norepinephrine reuptake inhibitor (NRI) and a carbonic anhydrase inhibitor (CAI) for use in treating a subject having a condition associated with central hypoventilation.

46. A therapeutic combination of (i) a norepinephrine reuptake inhibitor (NRI) and (ii) a carbonic anhydrase inhibitor (CAI) for use in treating a subject having a condition associated with central hypoventilation.
